(19) United States
(12) Patent Application Publication
(10) Pub. No.: US 2015/0100077 A1
(43) Pub. Date: Apr. 9, 2015

(54) THREAD FORMING JAMSHIDI ASSEMBLY

(71) Applicant: Safewire, LLC, Davie, FL (US)

(72) Inventor: Wyatt Drake Geist, Davie, FL (US)

(21) Appl. No.: 14/510,903

(22) Filed: Oct. 9, 2014

Related U.S. Application Data
(60) Provisional application No. 61/888,847, filed on Oct. 9, 2013.

(51) Int. Cl. A61B 17/70

(52) U.S. Cl. CPC A61B 17/7091
USPC 606/185

(57) ABSTRACT

The present invention is directed towards a Jamshidi assembly which can be used for surgical procedures, such as inserting a pedicle screw within a patient. The Jamshidi assembly comprises an inner cannulated member sized and shaped to receive a sharp pointed medical device and slidably disposed within an outer sleeve member. When engaged with the inner cannulated member, the outer sleeve member is constructed to rotate and move about a longitudinal axis of the Jamshidi assembly. Coupled to the outer sleeve member is a working end adapted to contact and be driven into an anatomical structure, such as a vertebral body. Depending on the working end, configurations, such as threading may be formed within the anatomical structure.

### PRIORITY CLAIM

In accordance with 37 C.F.R. 1.76, a claim of priority is included in an Application Data Sheet filed concurrently herewith. Accordingly, the present invention claims priority to U.S. Provisional Patent Application No. 61/888,847, entitled “THREAD FORMING JAMSHIDI ASSEMBLY”, filed Oct. 9, 2013. The contents of the above referenced application is incorporated herein by reference in its entirety.

### FIELD OF THE INVENTION

The present invention relates to surgical instruments used for spinal surgery; more particularly to a thread forming Jamshidi assembly used in spinal surgery.

### BACKGROUND OF THE INVENTION

The central nervous system, made primarily of the brain and the spine, is a vital part of the human physiology responsible for coordinating many aspects of human activity. The spinal cord is made up of a bundle of nerve tissue and acts as a conduit to communicate neuronal signals from the brain to the rest of the body. Protecting the spinal cord is the spinal, or vertebral, column. Anatomically, the spinal column is made up of several regions, including the cervical, thoracic, lumbar and sacral regions, each containing a plurality of vertebrae. Medical procedures involving the vertebrae are normally complicated because of the preciseness and accuracy required to avoid both neural damage and injury to major blood vessels.

While most people have fully functional spinal cords, it is not uncommon for individuals to suffer some type of spinal ailment. For example, spinal fractures, or vertebra compression fractures, occur when one of the bones of the spinal column fractures. Depending on the injury, various procedures have been developed to provide relief.

Many spinal surgical produces, such as minimally invasive percutaneous techniques, use various hardware devices, such as metallic screws, such as pedicle screws, rods, plates, or cages. U.S. Pat. No. 7,780,706 is an illustrative example of a pedicle screw assembly having a cannulated pedicle screw. As part of the surgical procedures, the pedicle screw gets passed over a previously placed guide wire. While such technique is relatively safe and effective, several possible problems are known to exist. For example, using a guide wire, while relatively safe, exposes the patient to risk of surgical complications. First, guide wires may advance through softer cancellous bone, resulting in severe damage to organs or vessels. Second, guide wires tend to travel a great distance during placement. Such increased distance can result in formation of kinks. Use of guide wires cause increased length of surgical instruments. The increased length makes the instruments more cumbersome, particularly when moving around fluoroscopic imaging devices, such as C-arm, which are critical for percutaneous screw instrumentation.

Accordingly, what is needed in the art is a device that can aid a surgeon in safely delivering pedicle screws to the patient.

### SUMMARY OF THE INVENTION

The present invention is directed towards a Jamshidi assembly which aids the user, i.e. a surgeon, in inserting a pedicle screw within a patient. The Jamshidi assembly comprises an inner cannulated member sized and shaped to receive a sharp pointed medical device, and an outer sleeve member. The inner cannulated member is slidably disposed within the outer sleeve member thereby forming a coaxial relationship. When engaged with the inner cannulated member, the outer sleeve member is constructed to rotate and/or move about a longitudinal axis of the Jamshidi assembly. Coupled to the outer sleeve member is a working end adapted to contact and be driven into an anatomical structure, such as a vertebral body. Depending on the working end, configurations, such as threading or a reamed opening, may be formed within the anatomical structure.

Accordingly, it is an objective of the present invention to teach a Jamshidi assembly which can be used to aid a user during a surgical procedure.

It is a further objective of the present invention to teach a Jamshidi assembly which can be used to aid a user during insertion of pedicle screws.

It is yet another objective of the present invention to teach a Jamshidi assembly which contains an outer sheath member adapted to rotate about an inner cannula member.

It is a still further objective of the present which contains an outer sheath member adapted to move about an inner cannula and/or a longitudinal axis in a linear manner

Other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is an illustrative example of a Jamshidi assembly in accordance with the present invention;

FIG. 2 is a side view of the Jamshidi assembly illustrated in FIG. 1;

FIG. 3 is a cross sectional view of the thread forming Jamshidi assembly taken along lines 2A-2A in FIG. 2;

FIG. 4 is an exploded view of the forming Jamshidi assembly;

FIG. 5A is a partial view of an illustrative working end of the Jamshidi assembly, illustrating an embodiment of the working end as a reamer;

FIG. 5B is an alternative embodiment view of a working end of the Jamshidi assembly;

FIG. 6 is an illustrative embodiment of the medical instrument having a sharply pointed end;

FIG. 7 is an illustrative embodiment of the outer sleeve member of the Jamshidi assembly;

FIG. 8 is a partial view of the outer sleeve member, illustrating an alternative embodiment of the working end having a tapered distal end;

FIG. 9 is a partial view of the distal end of the outer sleeve member, illustrating an alternative embodiment of the working end having a multiple crown end;

FIG. 10 is a partial view of the distal end of the outer sleeve member illustrating an alternative embodiment of the working end having a tap;

FIG. 11 is a partial view of an alternative embodiment of the Jamshidi assembly;

FIG. 12 is a schematic representation of the Jamshidi assembly inserted within a patient;

FIG. 13 is a schematic representation of the Jamshidi assembly in which the outer cannula is rotated, advancing in a linear manner about the inner cannula, thereby driving a working end into a vertebral body;

FIG. 14 is a schematic representation of the Jamshidi assembly in which the working member is removed from the vertebral body, thereby exposing a threading formed therein.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

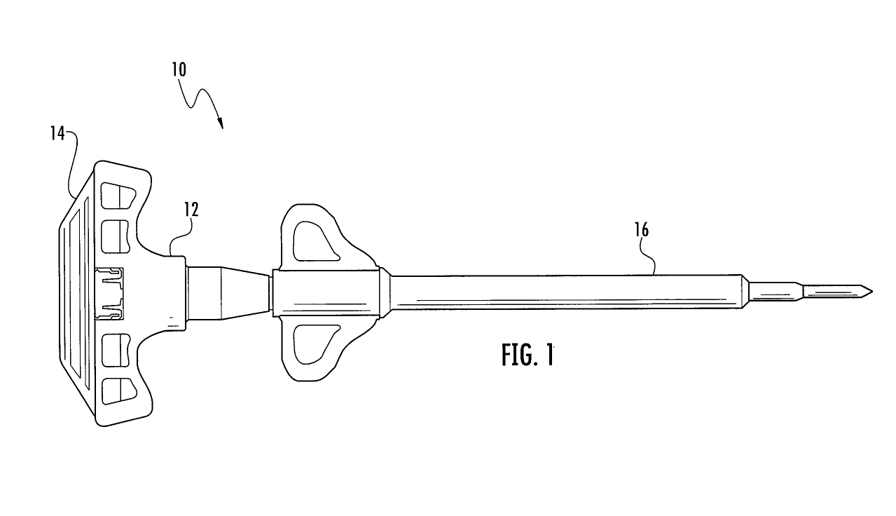

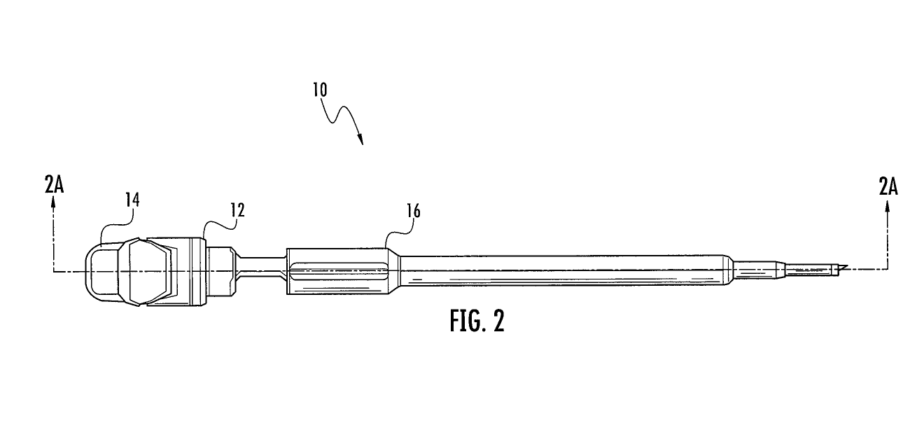

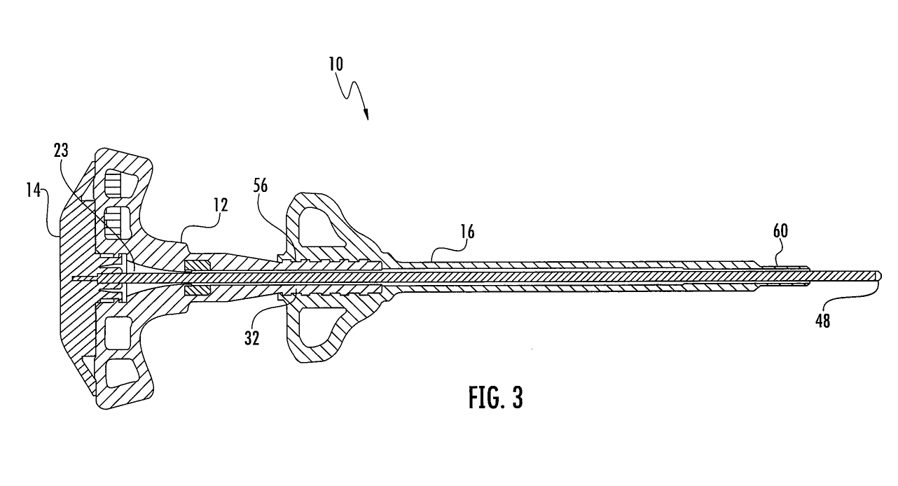

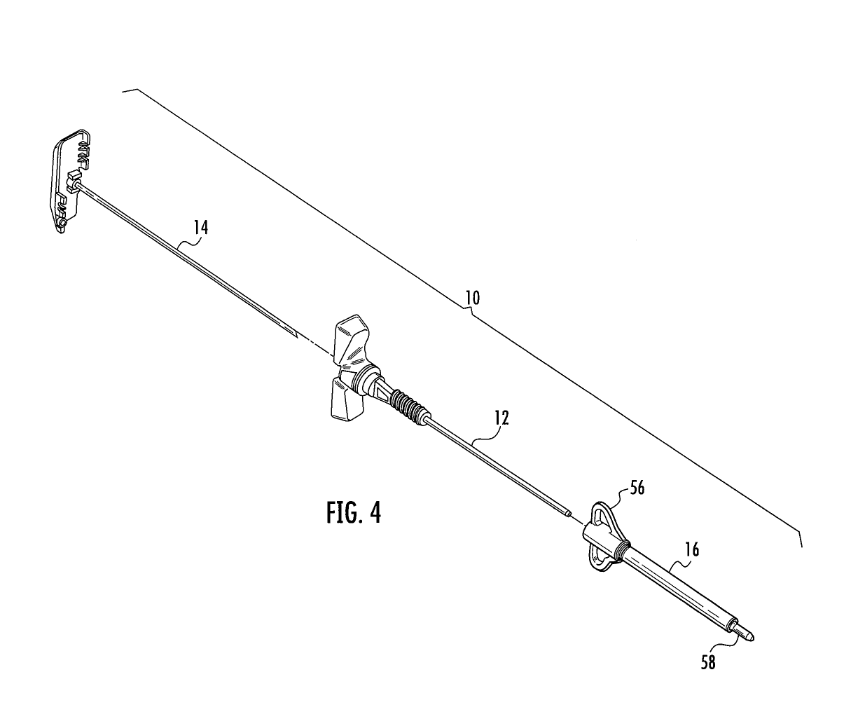

Referring to FIGS. 1-8, a Jamshidi type device having an outer sleeve with a working end, referred to generally as a Jamshidi assembly 10, is illustrated. The Jamshidi assembly 10 contains an inner cannulated member 12 sized and shaped to receive a sharp pointed medical device 14, and an outer sleeve member 16. The inner cannulated member 12 is slidably disposed within the outer sleeve member 16 thereby forming a coaxial relationship. When engaged with the inner cannulated member 12, the outer sleeve member 16 is constructed to rotate about and/or move linearly about a longitudinal axis of the Jamshidi assembly 10.

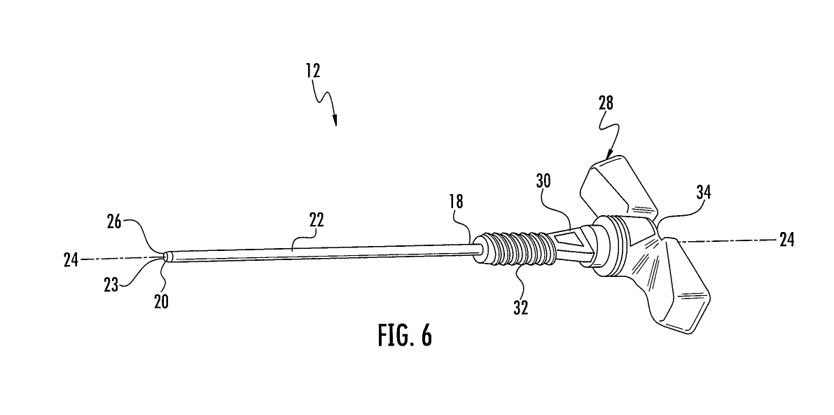

Referring to FIG. 6, the cannulated member 12 comprises a first end 18, a second end 20, and a main body 22. The main body 22 is preferably an elongated body having a generally cylindrical shape. Additionally, the main body 22 is cannulated having a hollow interior 23 sized and shaped to receive and allow passage of other devices, such as, for example, the sharp pointed medical device 14, or a guide wire (not shown), along its longitudinal axis 24. The second end 20 contains opening 26 which allows the other devices described above to extend out past the main body 22. Attached to the first end 18 is an inner cannulated member handle 28 adapted for gripping with a hand. The inner cannulated member handle 28 contains a collar 30 fixedly secured to the main body 22 as well as a threaded sleeve 32. The threaded sleeve 32 is designed to engage with a portion of the outer sleeve member 16. Preferably, the inner cannulated member handle 28 contains an ergonomic shape to provide a comfort fit for the user. The inner cannulated member handle 28 also contains opening 34 to allow for insertion and passage of various medical devices into and within the interior of the main body 22.

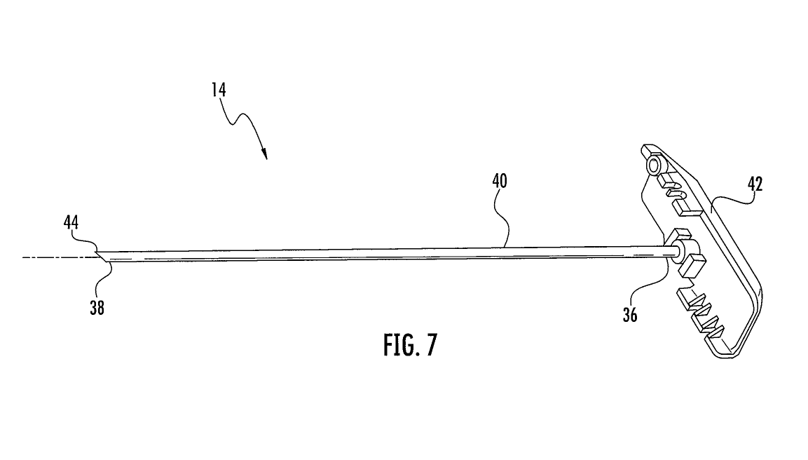

Referring to FIG. 7, an illustrative example of the sharp pointed medical device 14, herein as a stylet, is shown. The stylet 14 comprises a first end 38, a second opposing end 38, and a stylet elongated body 40 there between. The first end 36 contains a handle 42 which is constructed and arranged to be gripped by a user's hand. The handle 42 is also designed to engage and secure to the inner cannula member handle 28 so that when the stylet 14 is inserted into the interior, hollow portion 23 of the inner cannula member 12, it can be locked in place if required. The second opposing end 38 contains a pointed end, illustrated herein as a trocar-tapered stylet tip 44, adapted to puncture a body component, such as a vertebral body or other bone structure. The pointed end 44 may include for example, a standard pointed tip having two or more edges, or a single or multi-beveled tip, such as a bi-beveled or tri-beveled tip. Additionally, the second opposing end 38 may be constructed to contain threading.

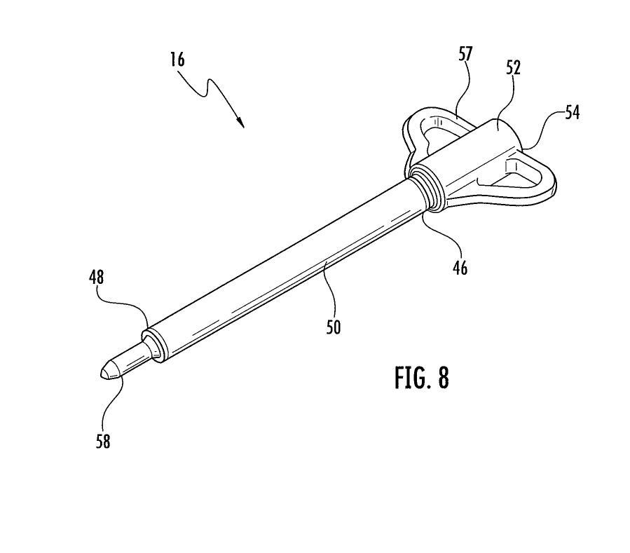

Referring to FIG. 8, an illustrative embodiment of the outer sleeve member 16 is illustrated. The outer sleeve member 16 contains a first end 46, a second end 48, and an outer sleeve member main body 50. The outer sleeve member main body 50 is preferably cannulated, having an interior hollow portion sized and shaped to slideably engage with the inner cannula member main body 22. Coupled to, or integrally formed with the first end 46 is an inner cannula member receiving portion, illustrated herein as a cylindrical body 52.

The cylindrical body 52 is sized and shaped to receive and engage with at least a portion of the inner cannulated member 12. Accordingly, the cylindrical body 52 contains opening 54 which exposes an interior portion that contains threading 56 (see FIG. 3). The threading 56 (preferably an internal, female threading) of the cylindrical body 52 provides a mechanism to engage with the threading (external, male threading) of the threaded sleeve 32. As such, when the outer sleeve member 16 slidably engages with the inner cannulated member 12 to form a coaxial relationship in which the threaded sleeve 32 engages with the threading 56, the outer sleeve member 14 can be linearly moved along a longitudinal axis in a controlled manner. To aid the user in accomplishing such movement, the cylindrical body 52 contains a handle 57 sized and shaped to allow a user to grip it and to provide user leverage when rotating the outer sleeve member 16. While the preferred embodiment of the outer sleeve member 16 contains threading 56, alternative embodiments of the Jamshidi assembly 10 may include an outer sleeve member 16 that does not contain the treading.

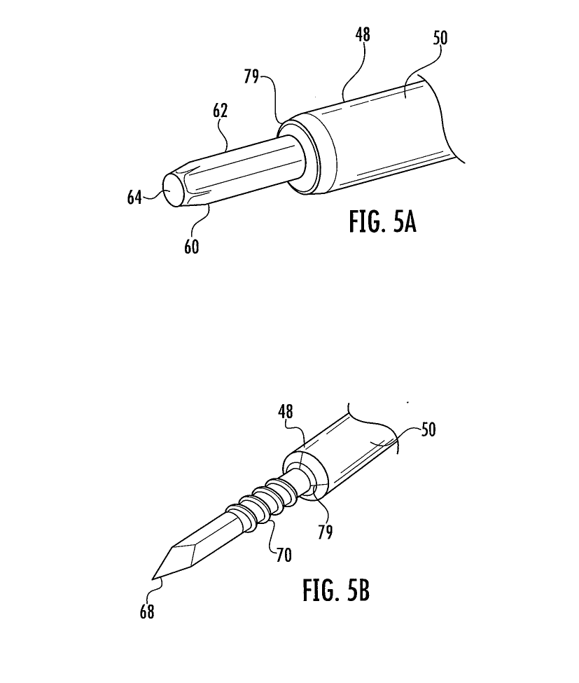

Positioned on the second end 48 is a working member 58. The working member 58 may be constructed as a separate, independent unit subsequently secured to the second end 48. Alternatively, the working member 58 can be integrally formed with and as part of the outer sleeve member main body 50. FIG. 5A shows an embodiment of the working member illustrated as a reamer 60 having strait flutes 62. Alternatively, the reamer 60 may contain spiral flutes or helical flutes. The reamer 60 contains opening 64 to allow the outer sleeve member 14 to move about the inner cannulated member 12. The leading edge and outer diameter of the flutes 62 are constructed to size the hole formed in the bone for tapping or for thread forming screws without he need to place a separate tool into the formed hole. Extended surface 79 provides a stop to prevent the tool from being over inserted into the bone. FIG. 5B illustrates an alternative embodiment of the working member 58. The working member 58 is shown as a tapping device comprising a pointed point end, such as a conical tip 68 and a threaded portion 70. In use, the conical tip 68 can be used to insert into a portion of the vertebral body and the threaded portion 70 can be used formation of conformations such as threading within the vertebral body, thereby aiding the insertion of a pedicle screw. Preferably, the thread pitch (distance from the crest of one thread to the next) and/or the lead (distance along the screw's axis that is covered by one complete rotation of the screw, 360 degrees) of the inner cannula threaded sleeve 32 is the same thread pitch and/or lead as threaded portion 70. In this manner, as the outer sleeve 16 is rotated, it moves along the inner cannula members 12 at the same rate or distance as the threaded portion 70 moves within the vertebral body. Alternatively, the threaded sleeve 32 may be designed to have different thread pitch and/or lead as that of the threaded portion 70. In addition, the threading 56 of the outer sleeve member may be constructed with the same thread pitch and/or lead as that of the working end 60 and/or other devices that contain threading, such as a tapping device in order to work in unison when in use. Alternatively, the threading 56 of the outer sleeve member 16 may have a different thread pitch and/or lead than the thread pitch of the working end 60 and/or other devices that contain threading.

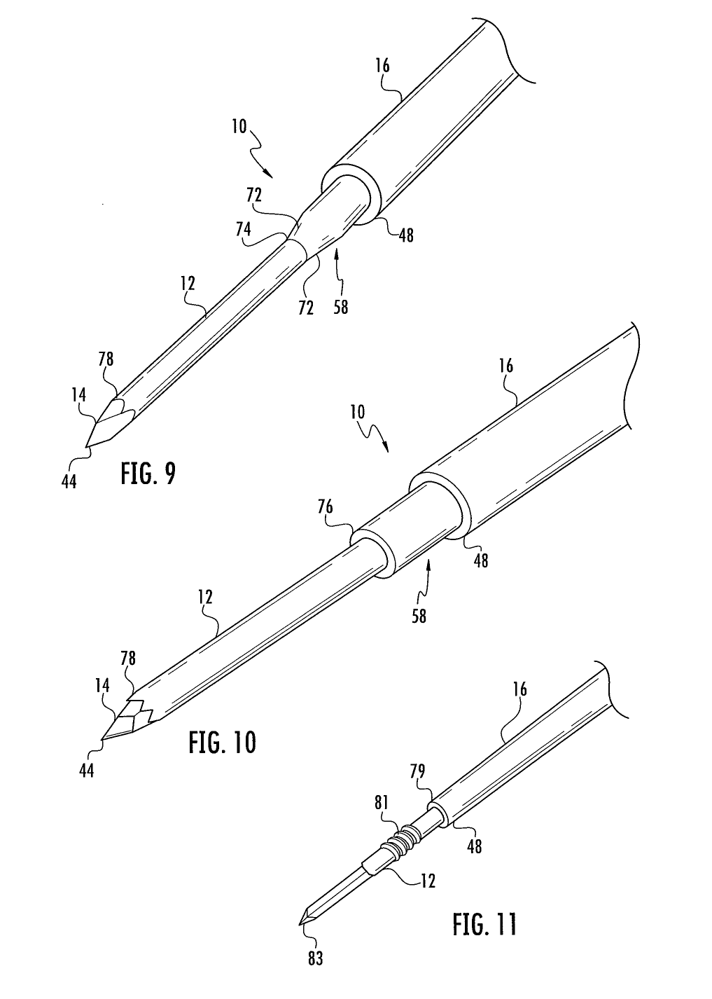

FIGS. 9 and 10 illustrate alterative embodiments of the Jamshidi assembly 10. The Jamshidi assembly 10 contains the same features as described above, differing mainly in the construction of the working member 58 of the outer sleeve member 16. As illustrated in FIG. 9, the working member 58 of the outer sleeve member 16 is shown having a tapered end 72 terminating in a generally smooth distal end surface 74. FIG. 10 illustrates the working member 58 of the outer sleeve member 16 having a triple crown cannula tip 76. The working member 58 may contain alternative surfaces having penetrating and/or advancement features, such as, but not limited to serrations or saw tooth ends. The triple crown cannula tip 76, along with the trocar-tapered stylet tip 44, and/or the multiple crown cannulated tip 78 of the inner cannula 12 provides a sharp, effective cutting edge designed for cortical penetration and medullary advancement which does not require much force from the surgeon.

FIG. 11 illustrate an alternative embodiment of the Jamshidi assembly 10. In the embodiment illustrated, the distal end 48 of the outer sleeve member 16 contains a tapered lip or extended surface 79 which can be used as a stop measure during insertion and/or removal. In addition, the Jamshidi assembly 10 is shown having inner cannula member 12 having threaded portion 81 and a pointed end 83. The threading can be designed to have the same thread pitch and/or lead as any thread pith and/or lead associated with other components of the Jamshidi assembly 10.

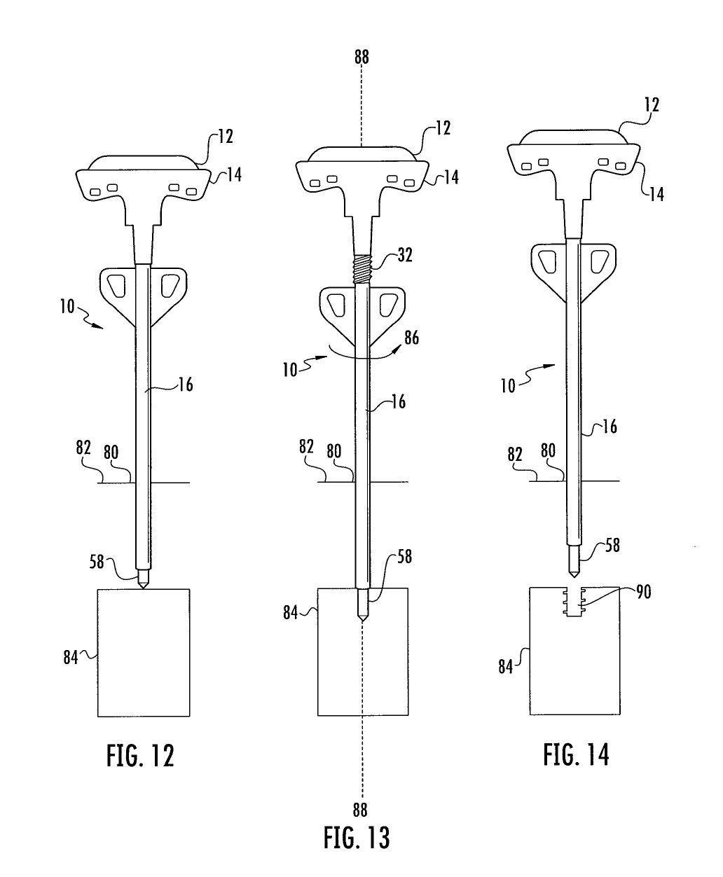

In use, the patient's skin and facia are incised according to standard medical protocol. The muscular tissues may be dissected as required. The Jamshidi assembly 10 is inserted into the incision and docked at the proper place. As illustrated in FIG. 12, outer sleeve member 16, the inner cannula 12, and/or the stylet 16 is passed through an incision 80 through a patient's skin 82, and rests along a portion of vertebral body 84 (for example, starting point for a pedicle screw). As the user rotates the outer sleeve member 16, see arrow 86, the outer sleeve member 16 rotates about the threaded sleeve 32 of the inner cannula 12. Such rotation allows the outer sleeve member 16 to traverse about the inner cannula 12 along the longitudinal axis 88 of the Jamshidi assembly. As the outer sleeve member 16 moves in a linear manner towards the vertebral body 84, the working end 58 eventually contacts the vertebral body 84. The outer sleeve member 16 is further rotated, driving the working end 58 into the vertebral body 84, see FIG. 13. The Jamshidi assembly 10 can be removed once the outer sleeve member 16 is rotated counterclockwise, back to its original starting point. Depending on the working end 58, some type of configuration, such as threading to aid in pedicle screw insertion, illustrated as partial dotted box 90 (see FIG. 14) is cut into the vertebral body 84.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A jamshidi assembly comprising:
an inner cannulated member including a first end, a second end, and a main body portion, an inner cannulated member handle adapted for gripping with a hand being secured to said first end thereof, said inner cannulated handle member including an opening positioned to allow insertion of various medical devices through an interior passage of said inner cannulated member, a threaded sleeve secured about said main body portion of said inner cannulated member,
a stylet including a first end, a second end and an elongated body there between, said first end including a handle portion constructed and arranged for attachment to said inner cannulated member handle, said elongated body sized to fit through said interior passage sufficiently for said first end of said stylet to protrude from said first end of said inner cannulated member when inserted through said second end thereof,
an outer sleeve member, said outer sleeve member including a first end, a second end and a main body portion, said outer sleeve member being cannulated to include an interior hollow portion, said interior hollow portion sized to slideably engage an outer diameter of said main body portion of said inner cannulated member, said second end of said outer sleeve member including a working member, said working member constructed and arranged to increase the size of an aperture created by said interior hollow portion.

2. The jamshidi assembly of claim 1 wherein said working member is a reamer.

3. The jamshidi assembly of claim 2 wherein said reamer includes at least one flute extending along the length thereof.

4. The jamshidi assembly of claim 3 wherein said at least one flute is straight.

5. The jamshidi assembly of claim 3 wherein said at least one flute is helical.

6. The jamshidi assembly of claim 1 wherein said working member is cylindrical in shape.

7. The jamshidi assembly of claim 1 wherein said working member is frustoconical in shape, a leading end of said working member being smaller in diameter, whereby said working member increases in diameter as inserted into a bone.

8. The jamshidi assembly of claim 1 wherein said outer sleeve member and said inner cannulated member include mating helical threads, whereby said outer sleeve member can be linearly moved along a longitudinal axis of said inner cannulated member in a controlled manner by rotating one of said outer sleeve member or said inner cannulated member.

9. The jamshidi assembly of claim 8 wherein said outer sleeve member includes a handle.

10. The jamshidi assembly of claim 1 wherein said outer sleeve member includes an extended surface, said extended surface constructed and arranged to engage an outer bone surface to provide a stop to prevent further insertion of said working member.

11. The jamshidi assembly of claim 8 wherein said working end is a tapping device constructed and arranged to form threads within bone tissue, said mating helical threads having a thread pitch that matches a thread pitch of said working member.

12. The jamshidi assembly of claim 1 wherein said working member includes a leading end said leading end including a serrated tip.

13. The jamshidi assembly of claim 1 wherein said serrations are saw teeth.

14. A jamshidi assembly comprising:
an inner cannulated member including a first end, a second end, and a main body portion, an inner cannulated member handle adapted for gripping with a hand being secured to said first end thereof, said inner cannulated handle member including an opening positioned to allow insertion of various medical devices through an interior passage of said inner cannulated member, a threaded sleeve secured about said main body portion of said inner cannulated member,
a stylet including a first end, a second end and an elongated body there between, said elongated body sized to fit through said interior passage sufficiently for said first end of said stylet to protrude from said first end of said inner cannulated member when inserted through said second end thereof,
an outer sleeve member, said outer sleeve member including a first end, a second end and a main body portion, said outer sleeve member being cannulated to include an interior hollow portion, said interior hollow portion threadably engaged to an outer diameter of said main body portion of said inner cannulated member, said second end of said outer sleeve member including a working member, said working member constructed and arranged to increase the size of an aperture created by said interior hollow portion.

15. A jamshidi assembly comprising any feature described, either individually or in any combination with any feature, in any configuration.
